(19) United States
(12) Patent Application Publication
(10) Pub. No.: US 2017/0296275 A1
(43) Pub. Date: Oct. 19, 2017

(54) TACE NAVIGATION GUIDANCE BASED ON TUMOR VIABILITY AND VASCULAR GEOMETRY

(71) Applicant: KONINKLIJKE PHILIPS N.V., EINDHOVEN (NL)

(72) Inventors: JULIUS CHAPIRO, BERLIN (DE); MING DE LIN, NEW HAVEN, CT (US); JEAN FRANCOIS GESCHWIND, EINDHOVEN (NL)

(21) Appl. No.: 15/515,742

(22) PCT Filed: Sep. 28, 2015
(86) PCT No.: PCT/IB2015/057414

§ 371 Date: Mar. 30, 2017

Related U.S. Application Data
(60) Provisional application No. 62/062,241, filed on Oct. 10, 2014.

(51) Int. Cl. A61B 34/20; A61B 6/00; A61B 6/12; A61B 5/055

(52) U.S. Cl. CPC A61B 34/20; A61B 6/5247; A61B 6/504; A61B 6/463; A61B 6/4085; A61B 6/12; A61B 5/055; A61B 2034/2065

(57) ABSTRACT

A system for transcatheter arterial chemoembolization (TACE) includes a visualization software module (115) configured to assess vascular geometry of an organ in an image of the organ. A tumor viability software module (124) is configured to provide a tumor viability map of the organ to be overlaid on the image of the organ. An imaging modality (126) is configured to track an instrument in or in proximity of the organ to ensure that the instrument is positioned within the organ for treatment in accordance with the tumor viability map.

This invention was made with government support under grant no. R01 CA160771-01 awarded by the National Cancer Institute of the United States National Institutes of Health. The government has certain rights in the invention.

### BACKGROUND

### Technical Field

This disclosure relates to medical imaging and more particularly to visualizing vascular geometry using overlaid tumor viability information in medical applications.

### Description of the Related Art

Given the fact that liver cancer (primary and metastatic) is primarily oxygen-supplied by the hepatic artery and is generally confined to the liver, drug delivery directly into the hepatic artery has been shown to be effective in the management of patients with this disease. Transcatheter arterial chemoembolization (TACE) is an x-ray image guided, interventional oncology procedure in which chemotherapeutic drug is delivered from a catheter in the hepatic artery. Level I evidence has demonstrated that patients have better symptom control and prolonged survival after TACE as compared to those receiving supportive care only (e.g., 5-year survival rate increases from 3% to 26%). This has resulted in TACE being the mainstay of intermediate stage hepatocellular carcinoma (HCC, primary liver cancer) therapy.

TACE patients are evaluated before and after a procedure with contrast-enhanced magnetic resonance imaging (MRI). The tumor response to treatment is routinely evaluated using contrast-enhancement based response criteria, which may include, e.g., the European Association for Study of the Liver (EASL) guidelines or modified Response Evaluation Criteria in Solid Tumors (mRECIST), etc. The tumor response is based on changes in the amount of enhancing tissue, as a measure of residual viable tumor. A developing observation of HCC and other solid tumors is that compared to healthy tissue, HCC exhibits an increase in the amount of blood vessels within the tumor as compared to healthy tissue, an increase of tortuosity and changes in overall vessel structure and density. The clinically observed blood vessel structure changes are further increased by embolization of the tumor feeding artery and can potentially cause technical difficulties for follow-up TACEs which could lead to insufficient tumor response.

### SUMMARY

In accordance with the present principles, a system for transcatheter arterial chemoembolization (TACE) includes a visualization software module configured to assess vascular geometry of an organ in an image of the organ. A tumor viability software module is configured to provide a tumor viability map of the organ to be overlaid on the image of the organ. An imaging modality is configured to track an instrument in or in proximity of the organ to ensure that the instrument is positioned within the organ for treatment in accordance with the tumor viability map.

A system for TACE includes a processor and memory coupled to the processor. The memory is configured to store a visualization software module configured to characterize and visualize vascular geometry of a region of interest, a tumor viability software module configured to intra-procedurally provide tumor viability imaging and viability-guided embolization with the vascular geometry of the region of interest and a prediction module configured to predict flow patterns, determine embolization endpoints and provide a feedback control mechanism for performing Sorafenib-treatment.

A method for TACE includes assessing vascular geometry of an organ in an image of the organ using a visualization software module; generating a tumor viability map of the organ to be overlaid on the image of the organ using a tumor viability software module; and determining embolization endpoints for an instrument in or in proximity of the organ to ensure that the instrument is positioned within the organ for treatment in accordance with the tumor viability map.

These and other objects, features and advantages of the present disclosure will become apparent from the following detailed description of illustrative embodiments thereof, which is to be read in connection with the accompanying drawings.

### BRIEF DESCRIPTION OF DRAWINGS

This disclosure will present in detail the following description of preferred embodiments with reference to the following figures wherein:

FIG. 1 is a block/flow diagram showing a system for transcatheter arterial chemoembolization (TACE) in accordance with one embodiment;

FIG. 2A shows a three dimensional (3D) image of a tumor viability map in accordance with the present principles;

FIG. 2B shows a two dimensional (2D) image of a tumor viability map in accordance with the present principles;

FIG. 2C shows another 2D image of a tumor viability map in accordance with the present principles;

FIG. 2D shows yet another 2D image of a tumor viability map in accordance with the present principles;

FIG. 3 is a model image showing a MIP-rendered qEASL viability map of a segmented tumor and tumor feeding arteries displayed in accordance with tumor viability information in accordance with the present principles;

FIG. 4 is a flowchart of an intra-procedural workflow showing integration of the present principles in a 3D visualization software application; and

FIG. 5 is a flow diagram showing a method for transcatheter arterial chemoembolization (TACE) in accordance with an illustrative embodiment.

### DETAILED DESCRIPTION OF EMBODIMENTS

In accordance with the present principles, systems and methods are provided for addressing insufficient or unselective tumor targeting, which can lead to incomplete tumor response. The present principles provide technical developments based on optimal blood vessel evaluation of a tumor during transcatheter arterial chemoembolization (TACE) and intra-procedural tumor viability information to address the need for quantitatively characterizing (i) blood vessel geometry and (ii) tumor viability. Specifically, the vessel geometry and tumor viability quantification are combined in an integration of a contrast-enhanced magnetic resonance imaging (MRI)/dual-phase cone beam computer tomography (CBCT) based semi-automated 3D-tumor viability and vessel geometry assessment software into 3D vessel visualization software. CBCT is cone beam computed tomography also referred to as C-arm CT, cone beam volume CT or flat panel CT. CBCT is a medical imaging technique including X-ray computed tomography where the X-rays are divergent, forming a cone.

The modifications in accordance with the present principles relate to identifying feeding arteries by adding target viability information to the profile of a selected tumor-feeding blood vessel. This builds upon the 3D vessel visualization software with the capability to measure and visualize vessel geometry parameters needed for the assessment of vascular geometry changes caused by various systemic and trans-arterial HCC treatments. The visualized vessel geometry parameters may include, e.g.: 1) Normalized Average Vessel Radius (NAVRAD); 2) Normalized Average Vessel Diameter (NAVD); 3) Normalized Vessel Count (NVC); 4) Vessel Segment Length (VSL); 5) Normalized Average Vessel Tortuosity by the Sum of Angles Metric (NSOAM); 6) Normalized Average Vessel Tortuosity by the Inflection Count Metric (NICM), etc.

Together, these modifications create a multi-level instrument with MRI-based tumor viability-guided target embolization, and dual-phase-CBCT based intra-procedural embolization endpoint assessment and vascular morphology response evaluation in patients treated with various TACE-based therapies.

TACE patients are evaluated before and after the procedure with contrast-enhanced MRI. The tumor response to treatment is routinely evaluated using three accepted methods for measuring changes in tumor size (e.g., Response Evaluation Criteria in Solid Tumors (RECIST)), enhancement (e.g., European Association for the Study of the Liver (EASL)), and tumor enhancement size (e.g., modified Response Evaluation Criteria in Solid Tumors (mRECIST)) on MR imaging. The EASL guideline is based on changes in the area of tumor enhancement on a representative slice, as a measure of residual viable tumor. Currently, it is being applied to one representative axial slice of the tumor. The assessment of enhancement percentage of the tumor area is based on visual inspection. Both, two-dimensional assessment as well as visual inspection, may lead to inaccuracy. A post-processing software module can produce semi-automatic three-dimensional segmentation and tumor viability measurements, based on contrast-enhanced MRI.

A developing observation of HCC and other solid tumors is that compared to healthy tissue, HCC exhibits an increase in the amount of blood vessels within the tumor as compared to healthy tissue, an increase of tortuosity and changes in overall vessel structure and density. The clinically observed blood vessel structure changes are further increased by embolization of the tumor feeding artery and can potentially cause technical difficulties for follow-up TACEs which could lead to insufficient tumor response.

Treatment strategies may include Sorafenib, a systemically administered drug, along with TACE. The combination of Sorafenib and TACE seems to improve overall survival among patients with advanced HCC compared to TACE alone. Sorafenib inhibits angiogenesis (growth of tumor blood vessels) and possibly alters the tumor vasculature. Specifically, this is through a phenomenon called vascular normalization, where vascular changes caused by the tumor reverse. There is growing evidence that the degree of vascular normalization can indicate therapy response. Currently, the method to assess vessel normalization is by visual inspection of angiograms.

Furthermore, a major limitation of systemic Sorafenib-treatment in TACE patients is the lack of therapy control. The systematic, quantitative, and standardized semi-automatic assessment of tumor vasculature in patients in accordance with the present principles represents an approach likely to change indications and drop-out criteria of the treatment. Moreover, the analysis of vascular geometry and hence the prediction of flow patterns is likely to improve the technical approach to chemoembolization and other trans-arterial therapies of HCC.

It should be understood that the present invention will be described in terms of medical instruments and systems; however, the teachings of the present invention are much broader and are applicable to other systems as well. In some embodiments, the present principles are employed in tracking and analyzing of complex biological or biomechanical systems. In particular, the present principles are applicable to internal tracking or treatment procedures for biological systems. The procedures may be in all areas of the body such as the liver, lungs, gastro-intestinal tract, excretory organs, blood vessels, etc. The elements depicted in the FIGS. may be implemented in various combinations of hardware and software and provide functions which may be combined in a single element or multiple elements.

The functions of the various elements shown in the figures can be provided through the use of dedicated hardware as well as hardware capable of executing software in association with appropriate software. When provided by a processor, the functions can be provided by a single dedicated processor, by a single shared processor, or by a plurality of individual processors, some of which can be shared. Moreover, explicit use of the term “processor” or “controller” should not be construed to refer exclusively to hardware capable of executing software, and can implicitly include, without limitation, digital signal processor (“DSP”) hardware, read-only memory (“ROM”) for storing software, random access memory (“RAM”), non-volatile storage, etc.

Moreover, all statements herein reciting principles, aspects, and embodiments of the invention, as well as specific examples thereof, are intended to encompass both structural and functional equivalents thereof. Additionally, it is intended that such equivalents include both currently known equivalents as well as equivalents developed in the future (i.e., any elements developed that perform the same function, regardless of structure). Thus, for example, it will be appreciated by those skilled in the art that the block diagrams presented herein represent conceptual views of illustrative system components and/or circuitry embodying the principles of the invention. Similarly, it will be appreciated that any flow charts, flow diagrams and the like represent various processes which may be substantially represented in computer readable storage media and so executed by a computer or processor, whether or not such computer or processor is explicitly shown.

Furthermore, embodiments of the present invention can take the form of a computer program product accessible from a computer-usable or computer-readable storage medium providing program code for use by or in connection with a computer or any instruction execution system. For the purposes of this description, a computer-usable or computer readable storage medium can be any apparatus that may include, store, communicate, propagate, or transport the program for use by or in connection with the instruction execution system, apparatus, or device. The medium can be an electronic, magnetic, optical, electromagnetic, infrared, or semiconductor system (or apparatus or device) or a propagation medium. Examples of a computer-readable medium include a semiconductor or solid state memory, magnetic tape, a removable computer diskette, a random access memory (RAM), a read-only memory (ROM), a rigid magnetic disk and an optical disk. Current examples of optical disks include compact disk—read only memory (CD-ROM), compact disk—read/write (CD-R/W), Blu-Ray™ and DVD.

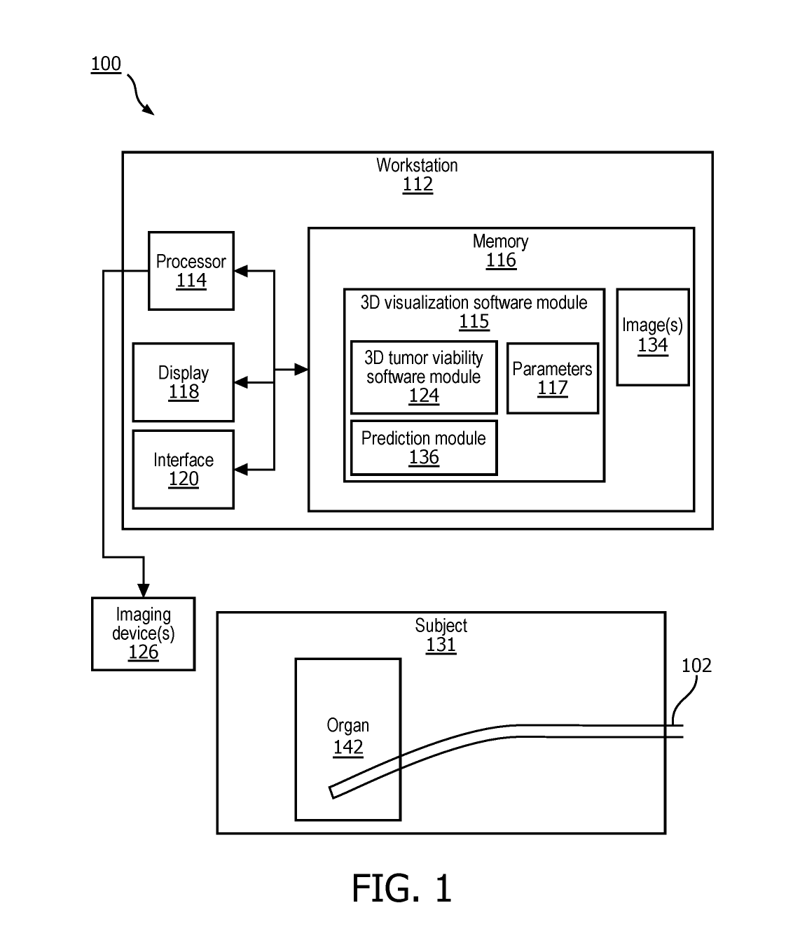

Referring now to the drawings in which like numerals represent the same or similar elements and initially to FIG. 1, a system 100 for transcatheter arterial chemoembolization (TACE) is illustratively shown in accordance with one embodiment. System 100 may include a workstation or console 112 from which a procedure is supervised and/or managed. Workstation 112 preferably includes one or more processors 114 and memory 116 for storing programs and applications. Memory 116 may store a 3D visualization software module 115 for characterization and visualization of vascular geometry, intra-procedural tumor viability imaging and viability-guided embolization, feedback/control of Sorafenib-Treatment and prediction of flow patterns and embolization endpoints, etc. Module 115 is configured to interpret measured data and images and to provide feedback to update visualizations of vasculature structures in a liver or other organ 142.

System 100 is configured to perform transcatheter arterial chemoembolization (TACE), which is a minimally invasive procedure performed in interventional radiology to restrict a tumor's blood supply. Small embolic particles coated with chemotherapeutic agents are injected selectively into an artery directly supplying a tumor. TACE is an interventional radiology procedure performed in an angiography suite. Percutaneous transarterial access is gained to the hepatic artery with an arterial sheath, e.g., by puncturing the femoral artery in the right groin and passing a catheter guided by a wire through the abdominal aorta, through the celiac trunk and common hepatic artery, and finally into the branch of the proper hepatic artery supplying the tumor. The interventional radiologist performs a selective angiogram of the celiac trunk and possibly the superior mesenteric artery to identify the branches of the hepatic artery supplying the tumor(s) and threads smaller, more selective catheters into such branches. This maximizes the amount of the chemotherapeutic dose that is provided to the tumor and minimizes the amount of the chemotherapeutic agent that could damage the normal liver tissue.

Alternating aliquots of the chemotherapy dose and of embolic particles, or particles including a chemotherapy agent, are injected through a catheter or other instrument 102. Agents introduced through that catheter may include Lipiodol, drug eluting particles, polyvinyl alcohol microspheres (doxorubicin), superabsorbent polymer microspheres (doxorubicin), gelatin microspheres (cisplatin), etc.

In one embodiment, workstation 112 includes a display 118 for viewing internal images of a subject (patient) or volume 131 and may include images 134 as an overlay or other rendering. Display 118 may also permit a user to interact with the workstation 112 and its components and functions, or any other element within the system 100. This is further facilitated by an interface 120 which may include a keyboard, mouse, a joystick, a haptic device, or any other peripheral or control to permit user feedback from and interaction with the workstation 112.

In accordance with the present principles, tumor viability quantification and vessel geometry are combined in module 115, which includes an integration of a contrast-enhanced MRI/dual-phase CBCT based semi-automated 3D-tumor viability and vessel geometry assessment software module 124 with 3D vessel visualization software of module 115. The automated 3D-tumor viability and vessel geometry assessment software module 124 may include quantitative EASL (qEASL) software.

Visualization software 115 is designed to plan an optimal access vascular pathway to a tumor and to predict ideal injection locations for the catheter 102 (e.g., a microcatheter) during TACE, using, e.g., intra-procedural CBCT imaging prior to 3D-segmentation-based reconstruction of a vascular tree, which is computed using the software module 124. In an intra-procedural part, qEASL-software module 124 employs a tumor viability approach, which includes a semi-automatic 3D tumor segmentation on contrast-enhanced MR imaging/contrast-enhanced CBCT scans. qEASL-software 124 based subtraction of pre-contrast MRI/CBCT images from the contrast-enhanced scan is employed to remove background enhancement. In accordance with the present principles, qEASL-software 124 based post-processing calculations, resulting in a quantitative 3D tumor viability map, is overlaid on the 3D vessel visualization tumor projection to show volumetric and regional/localized tumor enhancement heterogeneity, e.g., an imaging based marker for tumor viability.

Integration of target viability information (from module 124) is provided to the profile (from module 115) of a selected tumor-feeding blood vessel. The qEASL software 124 generates quantitative 3D viability maps which can be visualized using color-coded scales (e.g. from largely necrotic areas to highly viable tissues). One can use different visualization techniques as well. For example, simple 2D overlays, obtained as slices of the 3D viability map, maximum intensity projection (MIP) renderings for increased 3D depth perception, etc. MIP rendering of 3D volumes can be generated following any projection direction.

MIP renderings of the quantitative EASL (qEASL) 3D viability map are generated using a particular (known) orientation of the interventional imaging setup and overlaid, within the 3D visualization software module 115, along with feeding artery information. This modification changes the existing concept of feeding arteries by adding target viability information to the profile of a selected tumor-feeding blood vessel. Furthermore, 3D visualization software module 115 includes the capability of measuring and visualizing vessel geometry parameters needed for the assessment of vascular geometry changes caused by various systemic and trans-arterial HCC treatments.

Another part of the present principles is to build upon the 3D visualization software of module 115 with the capability to measure and visualize vessel geometry parameters 117, such as, e.g.:

1) Normalized Average Vessel Radius (NAVRAD, the sum of radii at all vessel skeleton points divided by the number of points. Results reported in mm. It may also be defined as the average radius of all vessel segments clipped to the region of interest.)

2) Normalized Average Vessel Diameter (NAVD, sum of average vessel diameters of a blood vessel segment divided by vessel length)

3) Normalized Vessel Count (NVC. The vessel count provides the number of individual, unbranched vessels contained within or passing through the region of interest and provides a measure of vessel density. When normalized (z-scored), a value of −1 indicates a count one standard deviation below the healthy mean and a value of 2.5 a count 2.5 standard deviations above the healthy mean.)

4) Vessel Segment Length (VSL, calculable for any segment chosen by the viewer)

5) Normalized Average Vessel Tortuosity by the Sum of Angles Metric (NSOAM, sums curvature along a space curve using successive trios of equally spaced vessel skeleton points and normalizes by vessel length. Values are reported as radians/cm. SOAM will be calculated for the whole hepatic vasculature, for the whole tumor vasculature and for a representative segment of the feeding artery. The SOAM value is almost invariably elevated for cancer-associated vasculature.)

6) Normalized Average Vessel Tortuosity by the Inflection Count Metric (NICM, calculates the number of “inflection” points along a space curve and multiplies this number (plus 1) by the total path length of the curve divided by the distance between endpoints. Inflection Count Metric (ICM) values are elevated when a curve exhibits a high amplitude sinusoidal pattern. Values reported as a dimensionless number. ICM will be calculated for the whole hepatic vasculature, for the whole tumor vasculature and for a representative segment of the feeding artery.)

One or more of these parameters 117 and others may be employed to create a standardized instrument for vascular response evaluation in patients treated with TACE and Sorafenib. Together, these parameters 117 may be employed to create a multi-level instrument with MRI-based tumor viability-guided target embolization, and dual-phase-CBCT based intra-procedural embolization endpoint assessment and vascular morphology response evaluation in patients treated with various TACE-based therapies. Other parameters and features may also be employed. For example, in one embodiment, a prediction module 136 is included to provide an estimation of the flow rate within the blood vessel. This may include using, e.g., the Navier-Stokes equation, the Hagen-Poiseuille equation and/or other equations of models. This aims to determine the embolization endpoint as much as to predict the flow patterns and the distribution of the embolic agent prior to releasing it from catheters located at bi- or trifurcations of lobar arteries and segment-feeding branches. The prediction module 136 is configured to predict flow patterns and determine embolization endpoints. The prediction module 136 includes a feedback control mechanism for performing Sorafenib-treatment based on the flow information and determined endpoints.

The information computed for each of these parameters may be graphically rendered in color showing intensity or density changes. Each parameter may be displayed alone or in combination with other parameters.

In another embodiment, the geometric vessel parameters can be employed to evaluate the accessibility of a vessel (e.g., length and diameter). In still another embodiment, the knowledge of vessel geometry permits the prediction of the type and size of instruments needed to achieve a particular result (e.g., stent sizing, selection of guide- and glide-wires, selection of micro-catheters, etc.).

Although the system 100 may employ stored images or models 134, the system 100 may also include imaging devices 126 (e.g., MRI, CBCT, etc.) for collecting images or making measurements employed by the visualization module 115 and/or the tumor viability module 124. In preferred embodiments, the imaging may be carried out at different times, in real-time (intra-procedural) or in different locations.

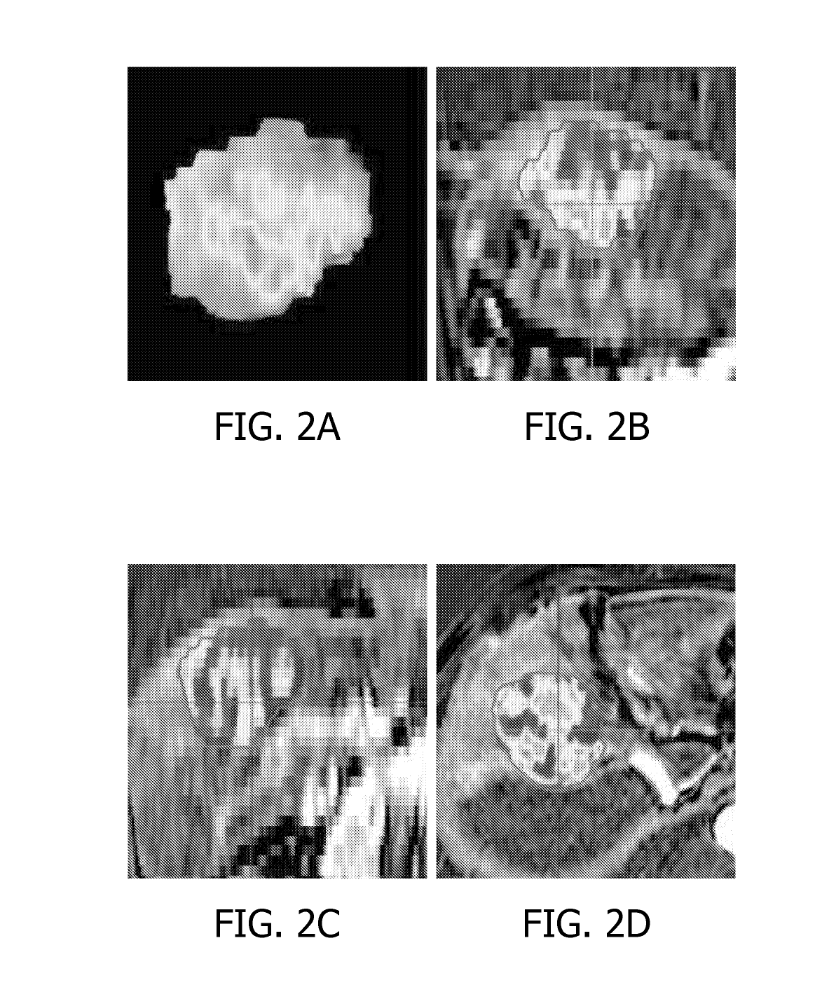

Referring to FIGS. 2A-2D, an illustrative visualization of qEASL 3D tumor viability maps in accordance with the present principles are shown. From the subtraction of pre-contrast MRI/CBCT images from the contrast-enhanced scan, the qEASL-software (124, FIG. 1) computes a 3D viability map within a tumor segmentation. This map can be visualized as a color-coded 3D Maximum Intensity Projection in arbitrary orientation (depicted in FIG. 2A) or as color-coded 2D overlays (depicted in FIGS. 2B, 2C and 2D).

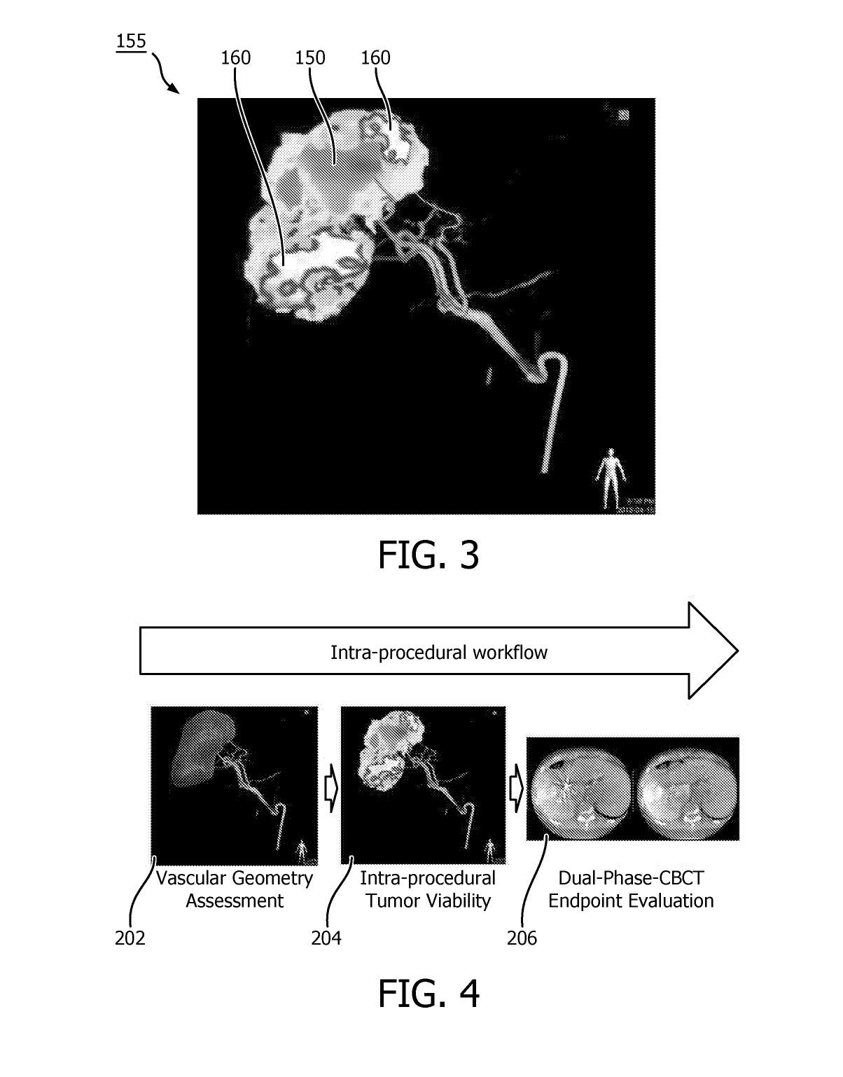

Referring to FIG. 3, a model image of the 3D visualization software application demonstrates a MIP-rendered qEASL viability map 155 of the segmented tumor and tumor feeding arteries displayed in accordance with the tumor viability information of the present principles. The blood vessel association with the concurrent tissue viability information is color-coded (e.g., red represents highly viable tissue 150 and the concurrent feeder, blue represents largely necrotic tissue 160).

Referring to FIG. 4, a flowchart of the intra-procedural workflow shows the integration of the present principles in a 3D visualization software application. This application is used to address the need to visualize tumor viability and vessel geometry to include intra-procedural tumor viability information into interventional radiology (IR) practice, to predict flow patterns, to provide embolization endpoints, to demonstrate vascular anatomy variations and to assess vessel compatibility with IR instruments.

In block 202, a vascular geometry assessment is performed. This may include collecting MRI images of the liver or other organ. Vascular assessment may be performed using other available tools as well. The blood vessels are defined or modeled in a visualization of the organ. In block 204, an overlay may be placed on the liver or other organ to show tumor viability. The tumor viability information is collected during the procedure (intra-procedure) and can demonstrate to an operator where chemo or other treatment materials should be provided. In block 206, end-point evaluation is performed. This may include the use of a dual-phase CBCT. In this way, guidance information is provided to a user regarding the placement of chemo dispensing devices. The operator will have the benefit of the tumor viability information on a display, and the instrument for dispensing chemo may be imaged along with the 3D visualization of the organ, the tumor viability information and the instrument. Predictive flow patterns may also be generated and provided in the image. This may be employed for planning a procedure or during a procedure.

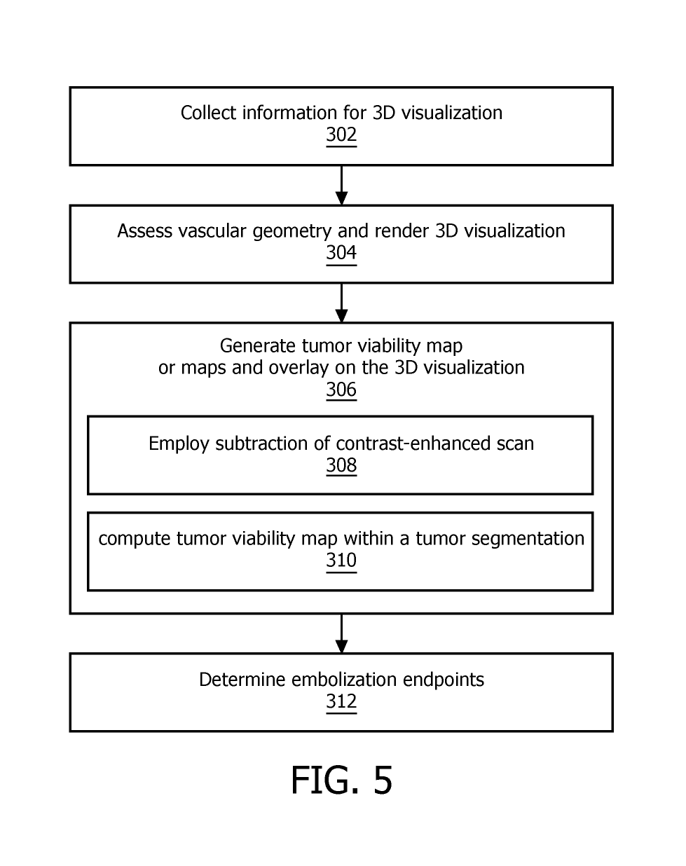

Referring to FIG. 5, a method for transcatheter arterial chemoembolization (TACE) is illustratively shown. In block 302, collect images and/or data of an organ for 3D visualization. In block 304, vascular geometry of an organ is assessed in an image (or model) of the organ using a visualization software module. The 3D image may be rendered or generated for display. The vascular geometry may include one or more of: Normalized Average Vessel Radius (NAVRAD) Normalized Average Vessel Diameter (NAVD), Normalized Vessel Count (NVC), Vessel Segment Length (VSL), Normalized Average Vessel Tortuosity by the Sum of Angles Metric (NSOAM) and/or Normalized Average Vessel Tortuosity by the Inflection Count Metric (NICM).

In block 306, a tumor viability map of the organ is generated to be overlaid on the image of the organ using a tumor viability software module. In block 308, the tumor viability map may include a subtraction of pre-contrast magnetic resonance images and cone based computed tomography (CBCT) images from a contrast-enhanced scan. In block 310, the tumor viability map may be computed within a tumor segmentation and visualized as one or more of a color-coded 3D Maximum Intensity Projection in arbitrary orientation or as a color-coded 2D overlay. The tumor viability maps may include color-coded scales from largely necrotic areas to highly viable tissues.

The tumor viability software module may include quantitative European Association for Study of the Liver (qEASL)-software based post-processing calculations to show volumetric and regional or localized tumor enhancement heterogeneity. The tumor viability software module may also include integration of target viability information to a profile of a selected tumor-feeding blood vessel.

In block 312, embolization endpoints are determined for an instrument in or in proximity of the organ to ensure that the instrument is positioned within the organ for treatment in accordance with the tumor viability map. This provides navigation guidance for the administering of chemo or other treatments. This may also include the prediction of blood flow to assist in the positioning of chemotherapy agents and other treatment materials (e.g., Sorafenib-treatment).

In interpreting the appended claims, it should be understood that:
a) the word “comprising” does not exclude the presence of other elements or acts than those listed in a given claim;
b) the word “a” or “an” preceding an element does not exclude the presence of a plurality of such elements;
c) any reference signs in the claims do not limit their scope;
d) several “means” may be represented by the same item or hardware or software implemented structure or function; and
e) no specific sequence of acts is intended to be required unless specifically indicated.

Having described preferred embodiments for TACE navigation guidance based on tumor viability and vascular geometry (which are intended to be illustrative and not limiting), it is noted that modifications and variations can be made by persons skilled in the art in light of the above teachings. It is therefore to be understood that changes may be made in the particular embodiments of the disclosure disclosed which are within the scope of the embodiments disclosed herein as outlined by the appended claims. Having thus described the details and particularity required by the patent laws, what is claimed and desired protected by Letters Patent is set forth in the appended claims.

1. A system for transcatheter arterial chemoembolization (TACE), comprising:
a visualization software module configured to assess vascular geometry of an organ in an image of the organ;
a tumor viability software module configured to provide a tumor viability map of the organ to be overlaid on the image of the organ; and
an imaging modality configured to track an instrument in or in proximity of the organ to ensure that the instrument is positioned within the organ for treatment in accordance with the tumor viability map.

2. The system as recited in claim 1, wherein the vascular geometry includes one or more of:
Normalized Average Vessel Radius (NAVRAD) Normalized Average Vessel Diameter (NAVD), Normalized Vessel Count (NVC), Vessel Segment Length (VSL), Normalized Average Vessel Tortuosity by the Sum of Angles Metric (NSOAM) and/or Normalized Average Vessel Tortuosity by the Inflection Count Metric (NICM).

3. The system as recited in claim 1, wherein the tumor viability map includes a subtraction of pre-contrast magnetic resonance image and cone based computed tomography (CBCT) images from a contrast-enhanced scan.

4. The system as recited in claim 1, wherein the tumor viability map computed within a tumor segmentation and visualized as one or more of a color-coded 3D Maximum Intensity Projection in arbitrary orientation or as a color-coded 2D overlay.

5. The system as recited in claim 1, wherein the tumor viability software module includes quantitative European Association for Study of the Liver (qEASL)-software based post-processing calculations to show volumetric and regional or localized tumor enhancement heterogeneity.

6. The system as recited in claim 1, wherein the tumor viability software module includes integration of target viability information to a profile of a selected tumor-feeding blood vessel.

7. The system as recited in claim 1, wherein the tumor viability map includes color-coded scales from largely necrotic areas to highly viable tissues.

8. A non-transitory computer readable storage medium comprising a computer readable program for transcatheter arterial chemoembolization (TACE), wherein the computer readable program when executed on a computer causes the computer to function as the visualization software module and the tumor viability software module of claim 1.

9. A system for transcatheter arterial chemoembolization (TACE), comprising:
a processor;
memory coupled to the processor, the memory configured to store:
a visualization software module configured to characterize and visualize vascular geometry of a region of interest;
a tumor viability software module configured to intra-procedurally provide tumor viability imaging and viability-guided embolization with the vascular geometry of the region of interest; and
a prediction module configured to predict flow patterns, determine embolization endpoints and provide a feedback control mechanism for performing Sorafenib-treatment.

10. The system as recited in claim 9, wherein the vascular geometry includes one or more of:
Normalized Average Vessel Radius (NAVRAD) Normalized Average Vessel Diameter (NAVD), Normalized Vessel Count (NVC), Vessel Segment Length (VSL), Normalized Average Vessel Tortuosity by the Sum of Angles Metric (NSOAM) and/or Normalized Average Vessel Tortuosity by the Inflection Count Metric (NICM).

11. The system as recited in claim 9, wherein the tumor viability imaging includes a subtraction of pre-contrast magnetic resonance image and cone based computed tomography (CBCT) images from a contrast-enhanced scan.

12. The system as recited in claim 9, wherein the tumor viability imaging is computed within a tumor segmentation and visualized as one or more of a color-coded 3D Maximum Intensity Projection in arbitrary orientation or as a color-coded 2D overlay.

13. The system as recited in claim 9, wherein the tumor viability software module includes quantitative European Association for Study of the Liver (qEASL)-software based post-processing calculations to show volumetric and regional or localized tumor enhancement heterogeneity.

14. The system as recited in claim 9, wherein the tumor viability software module includes integration of target viability information to a profile of a selected tumor-feeding blood vessel.

15. (canceled)

16. (canceled)

17. A method for transcatheter arterial chemoembolization (TACE), comprising:
assessing vascular geometry of an organ in an image of the organ using a visualization software module;
generating a tumor viability map of the organ to be overlaid on the image of the organ using a tumor viability software module; and
determining embolization endpoints for an instrument in or in proximity of the organ to ensure that the instrument is positioned within the organ for treatment in accordance with the tumor viability map.

18. (canceled)

19. (canceled)

20. (canceled)

21. (canceled)

22. (canceled)

23. (canceled)
